(19) United States
(12) Patent Application Publication
(10) Pub. No.: US 2003/0055385 A1
(43) Pub. Date: Mar. 20, 2003

(54) Displaceable syringe guard

(76) Inventors: Paul Schooler, Fraser, MI (US); Rottman Michael, West Bloomfield, MI (US)

Correspondence Address: CARLSON, GASKEY & OLDS, P.C., 400 WEST MAPLE ROAD, SUITE 350, BIRMINGHAM, MI, 48009, US

(21) Appl. No.: 09/955,700

(22) Filed: Sep. 18, 2001

(51) Int. Cl. A61M 5/32

(52) U.S. Cl. 604/220

(57) ABSTRACT

A structure is disclosed for holding medical sharp instruments within covers. Contrary to the prior art, the medical sharp instruments are held within the cover, both during use and non-use positions. Most preferably, some locking structure is provided which locks the medical sharp instrument within the cover at both the use and non-use positions.

### BACKGROUND OF THE INVENTION

This invention relates to a guard for a medical syringe wherein a cover remains with the syringe during use, and is displaceable to a guard position, and to a retracted use position.

Syringes are utilized in a variety of medical situations. As an example, syringes are utilized to provide a vehicle for injecting various medicines into a patient. Also, syringes can be utilized to provide communication between a patient's blood vein, and a vessel such as an intravenous supply package, or a blood test tube.

Recently, medical practitioners have become aware of the possibility of contamination from infected body fluids. A growing percentage of the population may be infected by communicable disease, such as HIV. Medical personnel are at risk, once a syringe has exposed body fluids, if the syringe then punctures the medical personnel's body.

For this reason, a number of techniques have been developed to provide for the disposal of any sharp object involved with medical uses. However, while the disposable of syringes has been addressed, the immediate protection of a syringe after use is still deficient.

At best, separate covers which are removed during use, and then placed over the sharp implement are known. However, medical personnel may puncture themselves while replacing the cover back onto the syringe after use. Thus, the known covers have undesirable characteristics, and do not fully address the potential problem of puncture of a medical personnel.

### SUMMARY OF THE INVENTION

In disclosed embodiments of this invention, a cover remains with a syringe, but is movable between guard and use positions. More preferably, the relative position of the syringe and the cover is such that structure holds the syringe relative to the cover at both the guard and use positions.

In a first embodiment, the syringe is of the type utilized to provide an injection to a patient through a needle. The syringe itself has a plunger movable within an outer body. The outer body is received in a cover body, and the syringe body carries a pin. A spring biases the syringe body relative to the cover body rearwardly to the non-use or guard position. In this position, the syringe needle is received within an opening in the cover such that the syringe needle is not exposed. Further, the pin in the syringe is received in a notch in a slot in the cover body that holds the syringe at this non-use position.

When medical personnel desire to use the syringe, the syringe is turned relative to the cover such that the pin moves out of this notch. The pin may then move along the slot to the extended use position. At the extended position, the pin is then moved into a second “use” notch. At this position, the syringe is held at a use position relative to the cover at which the needle extends from the cover. Thus, in this way, while the cover is always associated with the syringe, it does not interfere with the use of the syringe.

After use, the medical practitioner merely needs to turn the syringe for a relatively small extent, and the syringe will then be preferably biased back to the non-use position through a spring arrangement which will be described below.

In a second embodiment, the syringe is of the type typically utilized to provide a tap to a patient's blood vessel such as for providing communication of an intravenous fluid to the patient, or for removing a blood sample from the patient. A cover is received around a needle. Structure on the syringe body including a wing structure is generally biased to an outward position. The wings are received within a slot in the cover. The wings are biased to a naturally relaxed position which is beyond that which would be allowed by the cover slot. Thus, the wings hold the syringe at any location once a user releases the wings. The wings are thus utilized by biasing them to a retracted position which will allow movement of the syringe within the slot and the cover. Once the syringe is moved to its extended use position, the wings can be released. The wings then hold the syringe at the extended position. Once the communication between the syringe and the patient is done, the wings can be moved back to the retracted position, and the syringe moved within the slot back to the covered non-use position. Similar to the first embodiment, the structure between the cover and the syringe ensures that the syringe and the cover are always maintained together, and that the syringe is held at both the use and non-use positions within the cover.

These and other features of the present invention can be best understood from the following specification and drawings, following which is a brief description.

### BRIEF DESCRIPTION OF THE DRAWINGS

FIG. 1 is an exploded view of one embodiment.

FIG. 2 is a cross-sectional view.

FIG. 3 is a view similar to the FIG. 2 embodiment but showing a different position.

FIG. 4 is a perspective view.

FIG. 5 shows a second exploded embodiment.

FIG. 6 shows a second embodiment in its non-use position.

FIG. 7 shows a second embodiment in its use position.

### DETAILED DESCRIPTION OF A PREFERRED EMBODIMENT

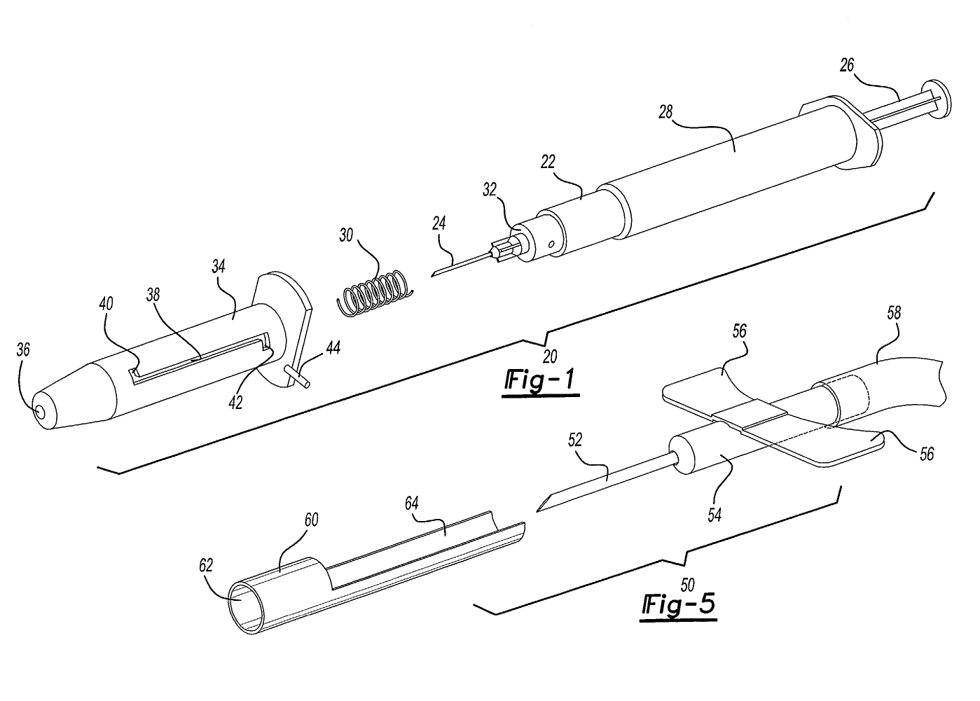

As shown in FIG. 1, an assembly 20 incorporates a syringe 22 including a needle 24 having a plunger 26 for moving the needle relative to a needle housing 28. The structure of the syringe 22 may be as known. A spring 30 biases the syringe 22 rearwardly relative to a cover 34. The spring 30 abuts an outer face 32 of the syringe housing 28. The cover 34 includes a forward opening 36 through which the needle 24 can selectively extend. Further, a slot 38 includes notches 40 and 42, as will be explained below.

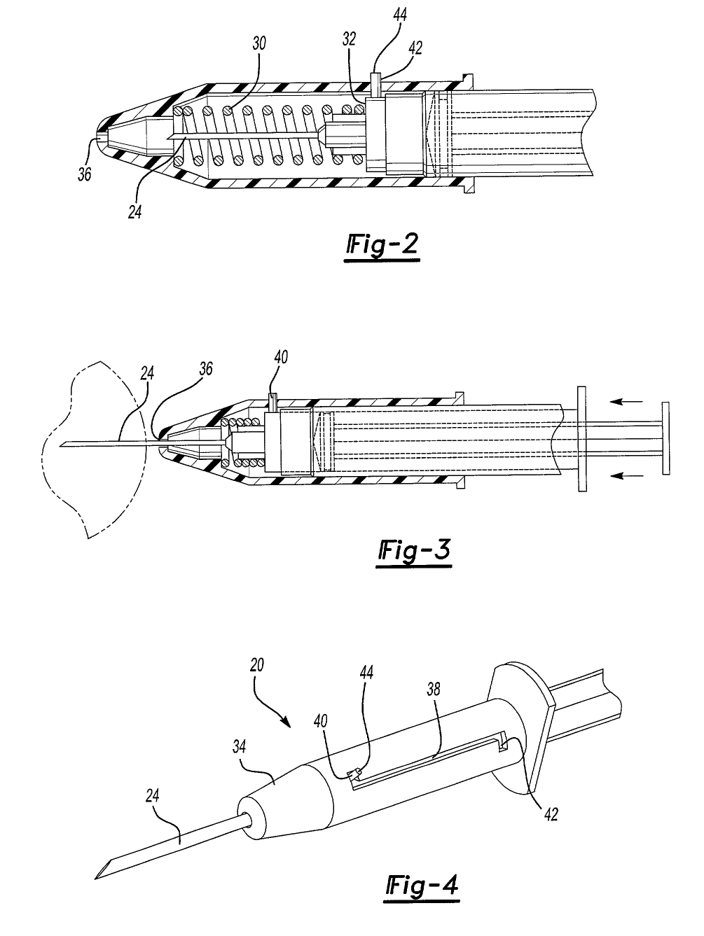

In FIG. 2, the spring 30 has biased the syringe 22 to a rearward non-use position. A pin 44 is shown within the notch 42, and holding the syringe 22 at its non-use position. As is apparent, needle 24 is protected within cover 34. As shown in FIG. 3, the syringe 22 has been slightly turned such that the pin 44 moves out of the notch 42 and may move along the slot 38. Once the pin 44 has been moved along the slot 38, such as applying a force in opposition to the spring 30, the pin 44 aligns with the notch 40. At this point, the syringe is turned and locked in the position such as shown in FIG. 3. Needle 24 extends outwardly of opening 36. At that point, a physician, or other medical personnel, may actuate the plunger 26 to achieve an injection. Once the injection is completed, the syringe body 22 is slightly rotated to move the pin 44 out of the notch 40, and allow the spring 30 to return the syringe body 22 to its non-use position.

As shown in FIG. 4, the pin 44 is received in the notch 40. As can be appreciated, once the pin 44 is rotated to be out of the notch 40, the spring will return the pin 44 through the slot 38 to the notch 42 unless a user is applying an opposed force.

This aspect of the invention thus provides a syringe wherein medical personnel are better protected in that the cover body 34 is always received with a syringe and is easily moved back to the non-use protected position such as shown in FIG. 2.

FIG. 5 shows an embodiment 50 wherein the syringe including needle 52 is arranged with a body 54 having wings 56. The wings 56 are such that they have an elastic free or relaxed position which will not be allowed by the size of a slot 64 on a cover 60. As shown, the needle 52 communicates with a flexible tube 58. The flexible tube 58 may be communicating to an intravenous package, or to a blood collection tube, etc. The cover 60 includes a forward opening 62.

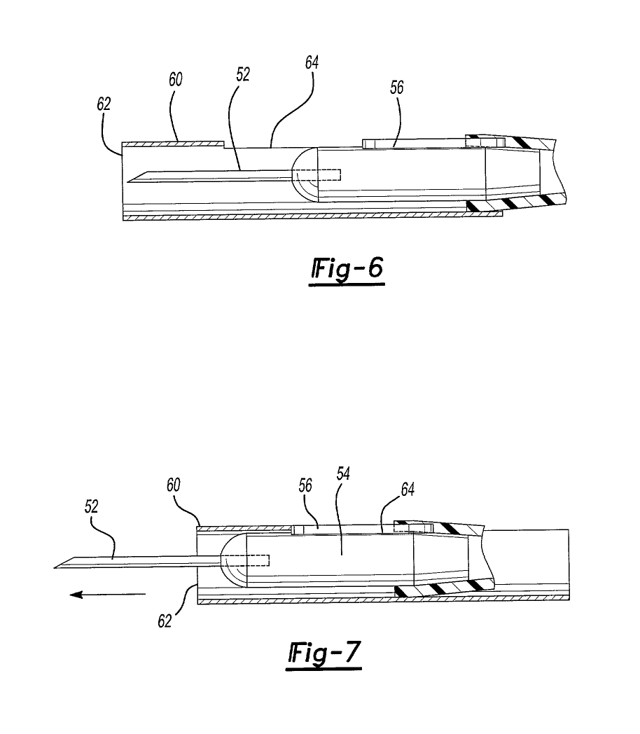

As shown in FIG. 6, the needle 52 is retracted such that it does not extend from the opening 62. The cover 60 thus protects the needle 52. The wings 56 are extending outwardly towards the relaxed position within the slot 64, however the wings preferably cannot reach their relaxed position such that they provide a bias force against the cover 60 holding the needle 52 at this retracted position. When a medical personnel desires to utilize the needle 52, the wings 56 are pivoted against this bias, such as being biased upwardly from the position shown in FIG. 5. The needle can then be moved forwardly along with the wings and body 54 to the position such as shown in FIG. 7. The wings are released, and again provides a bias force against the slot 64 holding the needle 52 forwardly. In this position, the needle can be utilized to provide an intravenous connection, or to obtain a blood sample.

The present invention thus provides a pair of embodiments wherein syringes for providing communication between a patient's bodily fluids can be protected by a cover which remains with the syringe during use and after use. Further, more preferably, the cover may be held on the syringe at both a use and non-use position. The prevent invention thus provides a safer cover for syringes.

Although preferred embodiments of this invention have been disclosed, a worker in this art would recognize that certain modifications would come within the scope of this invention. For that reason, the following claims should be studied to determine the true scope and content of this invention.

1. A medical device comprising:
a needle associated with a body;
a cover for selectively receiving said needle, said needle being movable relative to said cover between a use and a non-use position; and
structure on at least one of said body and said cover for holding said needle at both said use and non-use positions such that said needle can be selectively moved by medical personnel between said use and said non-use positions.

2. A medical device as set forth in claim 1, wherein said needle and said body are a syringe for injecting fluids into a patient, said syringe including a needle at a forward end and a plunger at a rearward end, and said syringe including a body received within a cover.

3. A medical device as set forth in claim 2, wherein said cover receives a spring for biasing said syringe to said non-use position.

4. A medical device as set forth in claim 3, wherein said syringe carries a pin which is slidable along a slot in said cover.

5. A medical device as set forth in claim 4, wherein said slot includes a non-use locked position and a use-locked position in said cover, and said pin being movable into said locked positions to hold said syringe relative to said cover at both said use and said non-use positions, respectively.

6. A medical device as set forth in claim 1, wherein said needle is associated with a body carrying a pair of biased wing portions, said wing portions having a relaxed position, and said cover including a slot with said body being movable within said slot, said wing portions not being able to extend to said relaxed position within said slot, and said wing portions holding said needle at both said use and non-use positions within said slot.

7. A syringe including:
a plunger for selectively injecting a needle into a patient, a syringe body allowing movement of said needle and said plunger relative to said syringe body; and
a cover receiving said syringe body and said needle, said cover including a spring biasing said syringe body to a non-use position, said syringe body including a pin movable within a slot in said cover such that said cover remains with said syringe during both use and non-use positions.

8. A medical device as set forth in claim 7, wherein said slot includes a non-use locked position and a use-locked position in said cover, and said pin being movable into said locked positions to hold said syringe relative to said cover at both said use and said non-use positions, respectively.

9. A syringe comprising:
a body, a needle and a wing structure such that said wing structure has a relaxed dimension; and
a cover for receiving said needle and said body, said cover including a slot having a predetermined dimension, said wings relaxed dimension being greater than said dimension of said slot such that when said body is received in said slot, said wings are not free to move to said relaxed dimension, and such that said needle is movable within said slot and relative to said cover between a use and non-use position with said wings holding said body in said slot.
